(19) United States
(12) Patent Application Publication
(10) Pub. No.: US 2022/0265875 A1
(43) Pub. Date: Aug. 25, 2022

(54) DISPLAY DISINFECTION DEVICE

(71) Applicant: LITEMAX ELECTRONICS INC., New Taipei City (TW)

(72) Inventors: SHANG-WEI LIU, New Taipei City (TW); YI-JHANG LIAO, New Taipei City (TW)

(21) Appl. No.: 17/179,443

(22) Filed: Feb. 19, 2021

(51) Int. Cl. A61L 2/10; A61L 2/24

(52) U.S. Cl. CPC A61L 2/10; A61L 2/24; A61L 2202/11; A61L 2202/14

(57) ABSTRACT

A display disinfection device includes a display, a disinfection module and a detection control module. The disinfection module is provided on the display for disinfecting a screen of the display. The detection control module is provided on the display and includes a controller, a first detector and a second detector. The first and second detector are electrically connected to the controller, such that the first detector and the second detector can respectively transmit a detection message to the controller after detecting a use state of the display, allowing the controller to determine according to the detection message to activate or to deactivate the disinfection module. In brief, the detection control module can activate the disinfection module to disinfect the display according to the use state of the display and to deactivate the disinfection module after a predetermined time period, allowing the display to be easily disinfected in real time.

### BACKGROUND OF THE INVENTION

### 1. Field of the Invention

The present disclosure relates to a display disinfection device, and in particular to a display disinfection device that provides real-time disinfection of a display and makes the display disinfection easier and more convenient to perform.

### 2. Description of the Related Art

Generally, an interactive display for a ticket sales system, a guide system, etc. enables users to operate on it by directly touching a screen of it. To maintain the display clean for use, the display screen is manually cleaned or sterilized when the display has been used over a period of time, so as to achieve the effect of disinfection.

However, the cleaning or sterilization of the display screen in the above mentioned way is usually manually done every several days or every several weeks that fails to provide real-time and effective disinfection.

### BRIEF SUMMARY OF THE INVENTION

An objective of the present disclosure is to overcome the shortage of the conventional way of display cleaning and sterilization by providing a display disinfection device, which enables real-time display disinfection and makes the display disinfection easier and more convenient than before.

To achieve at least the above objective, the display disinfection device according to the present disclosure includes a display, a disinfection module and a detection control module. The disinfection module is provided on the display for disinfecting a screen of the display. The detection control module is provided on the display and includes a controller, a first detector and a second detector. The controller is electrically connected to the display and the disinfection module, and the first and the second detector are electrically connected to the controller, such that the first detector and the second detector can respectively transmit a detection message to the controller after detecting a use state of the display, allowing the controller to determine according to the detection message to activate or to deactivate the disinfection module.

In the above display disinfection device, the display can be a flat display, a curved display, a round display or other geometrically shaped displays.

In the above display disinfection device, the display is a touch display.

In the above display disinfection device, the disinfection module can be an ultraviolet (UV) lamp provided on at least one of a top and a bottom of the display.

In the above display disinfection device, the disinfection module can include a plurality of ultraviolet (UV) lamps provided on a backlight module of the display, and the UV lamps are assembled to the display using a decorative cover each.

In the above display disinfection device, the disinfection module can be an ultraviolet (UV) lamp provided on at least one of two opposite sides of the display, and the UV lamp is assembled to the display using a decorative cover.

In the above display disinfection device, the controller is a central processor for computing and controlling the operation of the disinfection module, the first detector and the second detector.

In the above display disinfection device, the first detector is a human body infrared detector and the second detector is a timer.

With the above arrangements, the detection control module can activate the disinfection module to disinfect the display according to the use state of the display and deactivate the disinfection module after a predetermined time period of disinfection, so as to achieve the purpose of real-time disinfection of the display and make the display disinfection easier and more convenient than before.

### BRIEF DESCRIPTION OF THE DRAWINGS

FIG. 1 is a perspective view of a display disinfection device according to a preferred embodiment of the present disclosure.

FIG. 2 is an exploded view of FIG. 1.

FIG. 3 is a block diagram of the display disinfection device according to the preferred embodiment of the present disclosure.

### DETAILED DESCRIPTION OF THE INVENTION

To facilitate understanding of the objects, characteristics and effects of this present disclosure, embodiments together with the attached drawings for the detailed description of the present disclosure are provided. It is noted the present disclosure can be implemented or applied in other embodiments, and many changes and modifications in the described embodiments can be carried out without departing from the spirit of the disclosure, and it is also understood that the preferred embodiments are only illustrative and not intended to limit the present disclosure in any way.

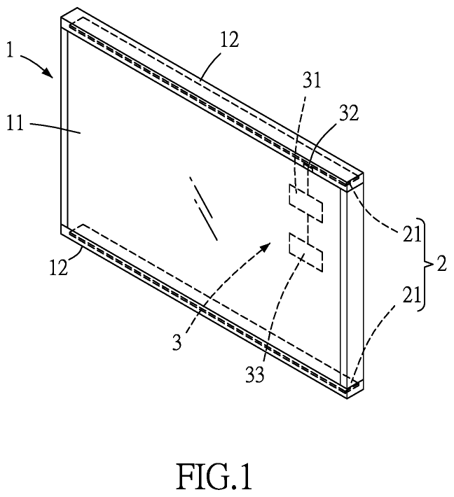

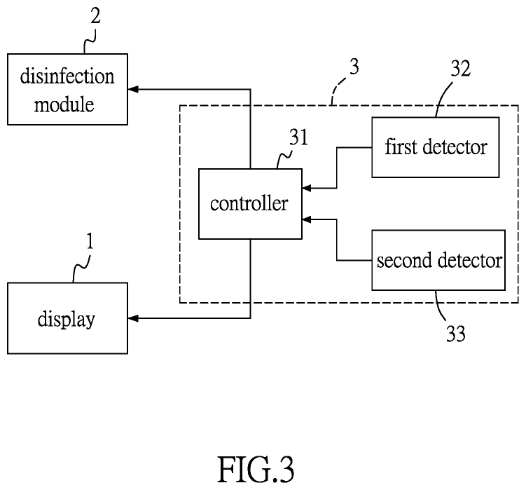

Please refer to FIGS. 1 to 3. As shown, the present disclosure provides a display disinfection device, which includes a display 1, a disinfection module 2 and a detection control module 3.

The display 1 can be a touch display in different configuration, such as a flat display, a curved display, a round display or other geometrically shaped display. In the illustrated preferred embodiment, the display 1 is a flat touch display.

The disinfection module 2 is provided on the display 1 to disinfect a screen 11 of the display 1.

The detection control module 3 is provided on the display 1 and includes a controller 31, a first detector 32 and a second detector 33. The controller 31 is electrically connected to the display 1 and the disinfection module 2. The first detector 32 and the second detector 33 are electrically connected to the controller 31, such that the first detector 32 and the second detector 33 can respectively transmit a detection message to the controller 31 after detecting a use state of the display 1, allowing the controller 31 to determine according to the detection message to activate or to deactivate the disinfection module 2.

In an embodiment of the present disclosure, the controller 31 is a central processor for computing and controlling the operation of the disinfection module 2, the first detector 32 and the second detector 33. The controller 31 is preferably a single chip, a logic circuit, or software.

When the display disinfection device is in use, the first detector 32 and the second detector 33 respectively detect the use state of the display 1. For example, the first detector 32 detects whether the display 1 is being used and transmits a detection message to the controller 32 after the latter has be used for a predetermined number of times. Then, the controller 31 determines according to the detection message from the first detector 32 to activate the disinfection module 2 for the latter 2 to disinfect the screen 11 of the display 1 and to deactivate the disinfection module 2 when the disinfection has been performed for a predetermined time period. On the other hand, it is also possible for the second detector 33 to detect a lapse of time and transmit a detection message to the controller 31 after a predetermined period of time has lapsed. Then, the controller 31 determines according to the detection message from the second detector 33 to activate the disinfection module 2 for the latter to disinfect the screen 11 of the display 1 and to deactivate the disinfection module 2 when the disinfection has been performed for the predetermined time period. In this way, according to the use state of the display 1, the detection control module 3 can activate the disinfection module 2 to disinfect the display 1 and deactivate the disinfection module 2 after the disinfection has been performed for a predetermined period of time, so that the display 1 can be disinfected in real time and in an easy and highly convenient manner.

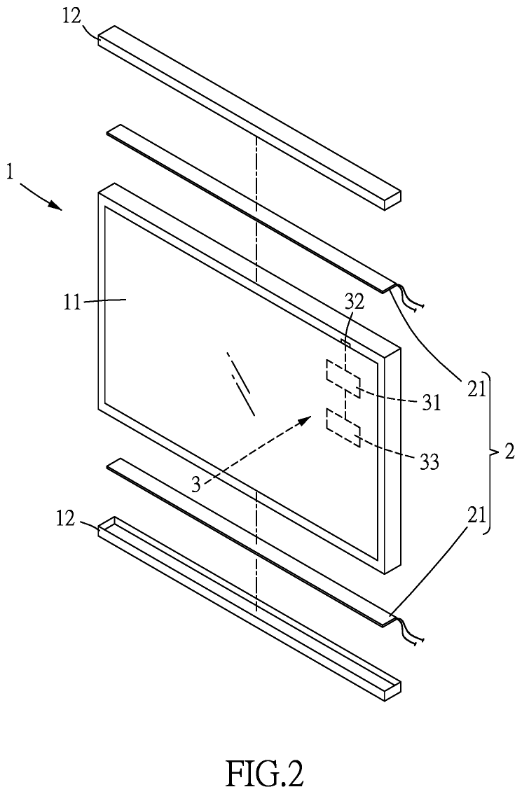

According to an embodiment of the present disclosure, the disinfection module 2 is an ultraviolet (UV) lamp 21 provided on at least one of a top and a bottom of the display 1, or a UV lamp 21 provided on at least one of two opposite sides of the display 1. Alternatively, the disinfection module 2 can be a UV lamp integrated with a backlight module (not shown) of the display 1. Further, the UV lamp 21 or the UV lamps 21 can be integrally formed in the display 1, or be additionally arranged on an outer side of the display 1. In the illustrated preferred embodiment, two UV lamps 21 are provided on the top and the bottom of the display 1, respectively. As can be seen in FIGS. 1 and 2, the two UV lamps 21 are respectively assembled to the top and the bottom of the display using a decorative cover 12 each. With this arrangement, the disinfection module 2 can radiate UV light on the screen 11 of the display 1 from both upper and lower sides of the display 1 to achieve the effect of completely disinfecting the screen 11 of the display 1.

According to an embodiment of the present disclosure, the first detector 32 is a human body infrared detector for detecting whether the display 1 is being operated or used by a user, and the second detector 33 is a timer for time detection. In this manner, the first detector 32 can detect whether the display 1 is being used and will transmit a detection message to the controller 31 after the display 1 has been used for a predetermined number of times. The controller 31 determines according to the detection message transmitted by the first detector 32 to activate the disinfection module 2 to disinfect the screen 11 of the display 1 and deactivates the disinfection module 2 when the disinfection has been performed for a predetermined period of time. Alternatively, it is also possible for the second detector 33 to detect time and transmit a detection message to the controller 31 when a predetermined length of time has lapsed. The controller 31 can determine according to the detection message transmitted by the second detector 33 to activate the disinfection module 2 to disinfect the screen 11 of the display 1 and deactivates the disinfection module 2 when the disinfection has been performed for a predetermined period of time. In this manner, the detection control module 3 can activate the disinfection module 2 to disinfect the display 1 according to the use state of the display 1 and deactivate the disinfection module 2 after a predetermined time period, so as to achieve the purpose of real-time disinfection of the display 1 and make the display disinfection easier and more convenient.

While the present disclosure has been described by means of specific embodiments, numerous modifications and variations could be made thereto by those skilled in the art without departing from the scope and spirit of the present disclosure set forth in the claims.

What is claimed is:

1. A display disinfection device, comprising:
a display;
a disinfection module being provided on the display for disinfecting a screen of the display; and
a detection control module being provided on the display and including a controller, a first detector and a second detector; the controller being electrically connected to the display and the disinfection module, and the first and the second detector being electrically connected to the controller, such that the first detector and the second detector can respectively transmit a detection message to the controller after detecting a use state of the display, allowing the controller to determine according to the detection message to activate or to deactivate the disinfection module.

2. The display disinfection device according to claim 1, wherein the display is selected from the group consisting of a flat display, a curved display, a round display and other geometrically shaped displays.

3. The display disinfection device according to claim 1, wherein the display is a touch display.

4. The display disinfection device according to claim 1, wherein the disinfection module is an ultraviolet (UV) lamp provided on at least one of a top and a bottom of the display, and the UV lamp being assembled to the display using a decorative cover.

5. The display disinfection device according to claim 1, wherein the disinfection module is an ultraviolet (UV) lamp provided on at least one of two opposite sides of the display, and the UV lamp being assembled to the display using a decorative cover.

6. The display disinfection device according to claim 1, wherein the disinfection module is an ultraviolet (UV) lamp provided on a backlight module of the display.

7. The display disinfection device according to claim 1, wherein the controller is a central processor for computing and controlling operations of the disinfection module, the first detector and the second detector.

8. The display disinfection device according to claim 1, wherein the first detector is a human body infrared detector and the second detector is a timer.
